(19) United States
(12) Patent Application Publication
(10) Pub. No.: US 2006/0159671 A1
(43) Pub. Date: Jul. 20, 2006

(54) Uses of sysnergistic bacteriophage lytic enzymes for prevention and treatment of bacterial infections

(76) Inventor: Vincent A Fischetti, Hempstead, NY (US)

Correspondence Address: Klauber & Jackson, 411 Hckensack Avenue, 4th Floor, Hackensack, NJ, 07601, US

(21) Appl. No.: 10/540,666

(22) PCT Filed: Dec. 23, 2003
(86) PCT No.: PCT/US03/41229

§ 371 Date: Mar. 8, 2006

Related U.S. Application Data
(60) Provisional application No. 60/436,077, filed on Dec. 23, 2002.

(51) Int. Cl. A61K 38/47; A61K 38/48

(52) U.S. Cl. 424/94.61; 424/94.63

(57) ABSTRACT

Methods for treating and preventing bacterial infections are described using at least two lytic enzymes obtained from a bacteriophage isolated from Streptococci. Two of these enzymes, Pal and Cpl-1, showed synergy when tested for cleavage of peptidoglycan in the cell walls of Streptococcus pneumoniae. Moreover, the combination of these two enzymes resulted in killing of both penicillin sensitive as well as penicillin resistant strains of S. pneumoniae. The synergy displayed by the combined use of these two enzymes may establish a means for identification of agents that mimic or enhance this activity and may lead to the identification of new antimicrobial agents and pharmaceutical compositions useful for treating and preventing a variety of bacterial infections in mammals. Further compositions are described for disinfecting or sanitizing porous and non-porous surfaces suspected of harboring infectious organisms.

### STATEMENT OF GOVERNMENT SUPPORT

The research leading to the present invention was supported in part by a grant from the Defense Advance Research Projects Agency and the Roche Research Foundation (DARPA Grant # DAMD19-010100318, Roche # Mkl/stm 95-2001). The government may have certain rights in the present invention.

### FIELD OF THE INVENTION

This invention relates to methods and compositions for the treatment of bacterial infections through use of a combination of bacteriophage lytic enzymes. The invention further relates to the use of this combination therapy for treatment of antibiotic resistant strains of bacteria. Furthermore, the invention relates to pharmaceutical compositions comprising a combination of bacteriophage lytic enzymes for use in treatment of bacterial infections.

### BACKGROUND OF THE INVENTION

Streptococcus pneumoniae colonize the nasopharynx in many adults and even more children (50% and up), where they are believed to have their reservoir (Robinson et al. 2001, JAMA 285(13): 1729-1735). Elimination or reduction of those colonizing organisms could have a major impact on the occurrence of local and systemic pneumococcal disease. New conjugate vaccines for children are being tested extensively and reveal a number of unsolved problems, such as the choice of included serotypes and serotype replacement in colonized children (Pelton et al. 2000, Vaccine 19 Suppl. 1:S96-99). The use of a substance that can specifically eliminate all serotypes of S. pneumoniae from the nasopharynx is likely to reduce the bacterial load in the community and enhance herd immunity in combination with a vaccine (Barbour et al. 1995, J. Infect. Dis. 171(1):93-98).

A substance has been identified that is able to accomplish this task. This substance, which is designated Pal, is an enzyme from a pneumococcal bacteriophage that can specifically digest any pneumococcal cell wall within seconds, resulting in rapid death of the organism (Loeffler et al. Science 294(5549):2170-2172).

Several such enzymes are known for pneumococcal bacteriophage, and are classified into two groups: Amidases, which cleave the peptidoglycan between N-acetylmuramic acid and L-alanine, and muramidases, such as lysozyme, which cleave the glycosidic bond between N-acetylmuramic acid and N-acetylglucosamine (Garcia et al. 1997, Microb. Drug Resist. 3(2):165-176). Cleavage with either of these enzymes results in a weakening in the cell wall, which leads to the externalization of the cytoplasmic membrane and ultimate lysis.

### SUMMARY OF THE INVENTION

In its broadest aspect the present invention provides for a method of treating or preventing bacterial infections, comprising administering to a subject in need of such therapy a therapeutically effective amount of a synergistic combination of at least two bacteriophage derived lytic enzymes and a suitable carrier for delivery of the lytic enzymes to the site of infection. It is a further object of the present invention to use a therapeutically effective synergistic combination of bacteriophage derived lytic enzymes for use in the treatment and prevention of bacterial diseases and infections, including those diseases and infections that are resistant to treatment with antibiotics such as, but not limited to, penicillin.

Accordingly, a first aspect of the invention provides for the identification of a combination of bacteriophage derived (eg. isolated from) lytic enzymes, which demonstrate synergistic bacteriocidal or bacteriostatic efficacy in vitro or in vivo, and the use of this combination for treatment or prevention of bacterial diseases or infections in subjects in need of such therapy. In a preferred embodiment, the infections to be treated are any infections caused by a bacterium whose cell wall contains peptidoglycan. These include both gram positive and gram negative organisms. In a preferred embodiment, the infections are caused by a streptococcus. In a further preferred embodiment, the infections are caused by Streptococcus pneumoniae. In a preferred embodiment, at least two synergistic bacteriophage derived lytic enzymes are used for the treatment or prevention of bacterial infections. In another preferred embodiment, at least three synergistic bacteriophage derived lytic enzymes are used for the treatment or prevention of bacterial infections. In yet another preferred embodiment, at least four synergistic bacteriophage derived lytic enzymes are used for the treatment or prevention of bacterial infections.

In another preferred embodiment, the at least two bacteriophage derived lytic enzymes are selected from the group consisting of an amidase, a muramidase, an endopeptidase, a glucosaminidase and combinations thereof. In another preferred embodiment, the lytic enzymes comprise an amidase and a muramidase and a suitable carrier to deliver the enzymes to the site of infection. In a preferred embodiment, the muramidase is lysozyme. In another preferred embodiment, the amidase is Pal and the lysozyme is Cpl-1. In another preferred embodiment, the lytic enzymes comprise an amidase and a glucosaminidase. In a yet further preferred embodiment, the lytic enzymes comprise an amidase and an endopeptidase. In yet another preferred embodiment, the amidase is Pal, which has a molecular weight (MW) of 34 kD and the lysozyme is Cpl-1, a phage lysozyme with a MW of 39 kD. When combined together, the two enzymes exhibit synergistic bacteriocidal or bacteriostatic activity in vitro and in vivo. Any synergistic combination of the enzymes described above may be used to treat or prevent bacterial infections.

In a further preferred embodiment, the infection to be treated is caused by Streptococcus pneumoniae. In a yet further preferred embodiment, the lytic enzymes decrease the occurrence or severity of local and systemic pneumococcal disease. In another preferred embodiment, the lytic enzymes prevent or eliminate pneumococcal colonization.

In another preferred embodiment, the at least two synergistic lytic enzymes are isolated from the phage of disease-causing bacteria. The lytic enzymes are preferably isolated from gram positive bacteria. More preferably, the lytic enzymes are isolated from Streptococci.

A second aspect of the invention provides for the in vitro use of at least two bacteriophage derived synergistic lytic enzymes to enhance the cleavage of the target bacterial peptidoglycan, which will ultimately result in destruction of the bacterial cell wall and hence show synergistic bacteriocidal or bacteriostatic activity.

Accordingly, in a preferred embodiment, the at least two bacteriophage derived synergistic lytic enzymes are selected from the group consisting of an amidase, a muramidase, an endopeptidase, a glucosaminidase and combinations thereof. Another preferred embodiment provides for a combination of at least two bacteriophage derived lytic enzymes, such as, but not limited to, those described herein, for decontaminating an area suspected of harboring infectious organisms such as streptococci. In a preferred embodiment, the at least two synergistic lytic enzymes includes an amidase and a muramidase. The muramidase may be lysozyme. In another preferred embodiment, the amidase is Pal and the lysozyme is Cpl-1. In another preferred embodiment, the at least two synergistic enzymes may be an amidase and a glucosaminidase. In a yet further preferred embodiment, the at least two synergistic lytic enzymes may include an amidase and an endopeptidase. In a yet further preferred embodiment, the combination may comprise at least three synergistic lytic enzymes. In a yet further preferred embodiment, the combination may comprise at least four synergistic lytic enzymes. Such use of a combination of lytic enzymes would prove effective in a hospital setting, wherein areas suspected of harboring infectious disease-causing organisms might be prevalent.

A further preferred embodiment of the present invention includes use of a combination of at least two synergistic bacteriophage lytic enzymes for the decontamination of inanimate objects and surfaces, for example, surgical instruments. Although the effective use of Pal and Cpl-1 are described herein as an effective combination of at least two lytic enzymes having such synergistic activity, this invention is not construed to be limited to the combination of lytic enzymes described herein.

A third aspect of the invention provides for a method of treatment or prevention of bacterial infections in vivo comprising administering a therapeutically effective amount of a synergistic combination of at least two bacteriophage derived lytic enzymes to cleave the bacterial peptidoglycan, such treatment resulting in more efficient prevention and elimination of bacterial colonization. Correspondingly, the present invention extends to the use of the lytic enzymes of the invention, and combinations thereof, for the preparation of a composition for the treatment of bacterial infections in vivo.

A preferred embodiment provides for a method of treatment or prevention of bacterial infections in vivo comprising administering a combination of at least two bacteriophage derived synergistic lytic enzymes, such as, but not limited to, those described herein, for cleaving the bacterial peptidoglycan, such cleaving resulting in lysing of the bacterial cell wall resulting in death or killing of the microorganism and more efficient prevention and elimination of bacterial colonization. In another preferred embodiment, the at least two bacteriophage derived lytic enzymes are selected from the group consisting of an amidase, a muramidase, an endopeptidase, a glucosaminidase and combinations thereof. In another preferred embodiment, the at least two synergistic lytic enzymes includes an amidase and a muramidase. The muramidase may be lysozyme. In another preferred embodiment, the amidase is Pal and the lysozyme is Cpl-1. Furthermore, the use of a combination of Pal and Cpl-l for in vivo therapy may prove to be effective in situations wherein the organisms prove to be resistant to standard antimicrobial therapies. In another preferred embodiment, the at least two synergistic enzymes may be an amidase and a glucosaminidase. In a yet further preferred embodiment, the at least two synergistic lytic enzymes may include an amidase and an endopeptidase. In a yet further preferred embodiment, the combination may comprise at least three synergistic lytic enzymes. In a yet further preferred embodiment, the combination may comprise at least four synergistic lytic enzymes.

A fourth aspect of the invention provides for the use of at least two therapeutically effective synergistic bacteriophage derived lytic enzymes for treatment and prevention of Streptococcus pneumoniae infections in vivo, including penicillin-resistant strains, and to provide for synergistic killing of the bacteria, and for more efficient prevention and elimination of bacterial colonization. It may be envisioned that the delivery of the lytic enzymes may either be by simultaneous administration, or may be in seriatim.

A preferred embodiment provides for a combination of at least two bacteriophage derived lytic enzymes, such as, but not limited to, those described herein, for treating or preventing bacterial infections in vivo, particularly penicillin resistant strains and for better elimination of bacterial colonization. In a preferred embodiment, the bacterial infection to be treated is a streptococcal infection. In a further preferred embodiment, the infection to be treated is a Streptococcus pneumoniae infection in vivo. In another preferred embodiment, the at least two bacteriophage derived lytic enzymes are selected from the group consisting of an amidase, a muramidase, an endopeptidase, a glucosaminidase and combinations thereof. In another preferred embodiment, the at least two synergistic lytic enzymes includes an amidase and a muramidase. The muramidase may be lysozyme. In another preferred embodiment, the amidase is Pal and the lysozyme is Cpl-l. Furthermore, the use of a combination of Pal and Cpl-1 for in vivo therapy may prove to be effective in situations wherein the Streptococcus pneumoniae organisms prove to be resistant to standard antimicrobial therapies. In another preferred embodiment, the at least two synergistic enzymes may be an amidase and a glucosaminidase. In a yet further preferred embodiment, the at least two synergistic lytic enzymes may include an amidase and an endopeptidase. In a yet further preferred embodiment, the combination may comprise at least three synergistic lytic enzymes. In a yet further preferred embodiment, the combination may comprise at least four synergistic lytic enzymes.

A fifth aspect of the invention provides for pharmaceutical compositions comprising at least two therapeutically effective synergistic bacteriophage derived lytic enzymes for treatment and prevention of local and systemic bacterial infections, and to provide for efficient prevention and elimination of bacterial colonization through use of these pharmaceutical compositions. Correspondingly, the present invention extends to the use of the lytic enzymes of the invention for the preparation of a composition for the prevention and elimination of bacterial colonization.

A preferred embodiment provides for inclusion of a combination of at least two bacteriophage derived lytic enzymes, such as, but not limited to, those described herein, in the pharmaceutical compositions along with pharmaceutically acceptable carriers for treatment and prevention of local and systemic bacterial infections, and to provide for efficient prevention and elimination of bacterial colonization. In a preferred embodiment, the local and systemic bacterial infections to be treated with the compositions of the present invention are any bacteria having peptidoglycan in their cell wall, including but not limited to, streptococcal organisms. In a further preferred embodiment, the streptococcal infections to be treated are caused by Streptococcus pneumoniae. In another preferred embodiment, the at least two bacteriophage derived lytic enzymes are selected from the group consisting of an amidase, a muramidase, an endopeptidase, a glucosaminidase and combinations thereof. In another preferred embodiment, the at least two synergistic lytic enzymes includes an amidase and a muramidase. The muramidase may be lysozyme. In another preferred embodiment, the amidase is Pal and the lysozyme is Cpl-1. In yet another preferred embodiment, the at least two synergistic enzymes may be an amidase and a glucosaminidase. In a yet further preferred embodiment, the at least two synergistic lytic enzymes may include an amidase and an endopeptidase. In a yet further preferred embodiment, the combination may comprise at least three synergistic lytic enzymes. In a yet further preferred embodiment, the combination may comprise at least four synergistic lytic enzymes.

In one specific embodiment, the pharmaceutical composition comprising the combination of lytic enzymes, including, but not limited to Pal and Cpl-1, may take into account the nature of the infection, and will determine whether the best mode for delivery to the site of the infection is via intravenous delivery, intramuscular delivery, subcutaneous delivery, or other means for providing the most effective and expeditious treatment of the infection.

A sixth aspect of the invention provides for an anti-microbial composition for sanitizing or decontaminating porous or non-porous surfaces comprising at least two bacteriophage derived synergistic lytic enzymes. In another preferred embodiment, the at least two bacteriophage derived lytic enzymes are selected from the group consisting of an amidase, a muramidase, an endopeptidase, a glucosaminidase and combinations thereof. In another preferred embodiment, the at least two synergistic lytic enzymes includes an amidase and a muramidase. The muramidase may be lysozyme. In another preferred embodiment, the amidase is Pal and the lysozyme is Cpl-1. In another preferred embodiment, the at least two synergistic enzymes may be an amidase and a glucosaminidase. In a yet further preferred embodiment, the at least two synergistic lytic enzymes may include an amidase and an endopeptidase. In a yet further preferred embodiment, the combination may comprise at least three synergistic lytic enzymes. In a yet further preferred embodiment, the combination may comprise at least four synergistic lytic enzymes.

A further embodiment of the invention provides for an antimicrobial composition suitable for decontaminating inanimate solid surfaces suspected of containing infectious bacteria by treating the surfaces with a bacteriocidal or bacteriostatically effective amount of the antimicrobial composition. Correspondingly, the present invention extends to the use of the lytic enzymes of the invention for the preparation of a composition for the decontamination of inanimate objects and surfaces.

Another preferred embodiment provides for an antimicrobial composition comprising at least two synergistic bacteriophage derived lytic enzymes useful for decontaminating inanimate objects, such as surgical instruments or other equipment used in various treatment regimens, such as dialysis equipment, in a hospital setting, prior to use of these objects or instruments for treating patients.

A preferred embodiment is a composition including bacteriostatic or bacteriocidal amounts of a combination of at least two synergistic lytic enzymes, including but not limited to an amidase and a muramidase, including but not limited to Pal and Cpl-1 for decontaminating inanimate surfaces, including but not limited to surgical instruments, dialysis equipment or other such equipment to be used in a situation whereby a patient may be exposed to infectious organisms.

Another preferred embodiment provides for a composition comprising the combination of at least two synergistic bacteriophage lytic enzymes useful for sanitizing or decontaminating porous surfaces e.g. textiles, carpeting. Furthermore, the composition of the at least two bacteriophage lytic enzymes may be used to decontaminate veterinarian surgical or examination areas, where such areas may be thought to harbor infectious organisms susceptible to the bacteriostatic or bacteriocidal activity of the combination of at least two bacteriophage derived enzymes.

In a further preferred embodiment, the at least two bacteriophage derived lytic enzymes may be combined with other bacteriostatic or bacteriocidal agents useful for decontamination of inanimate solid surfaces suspected of containing infectious bacteria, or for decontamination of porous surfaces.

A seventh aspect of the invention provides for a screening method for identifying agents capable of enhancing the activity of Pal and Cpl-1, comprising:

(a) preparing purified Pal and Cpl-1;

(b) contacting the Pal and Cpl-1 to a bacteria having radioactively labeled peptidoglycan in the cell wall in the presence or absence of a test compound under conditions which allow binding to the peptidoglycan; and

(c) determining the amount of peptidoglycan cleavage, wherein an agent capable of enhancing Pal and Cpl-1 activity is identified when the release of radioactivity is enhanced in the presence but not the absence of the agent.

In a preferred embodiment, the enhancer of Pal and Cpl-1 activity identified by the above noted method may be used to treat or prevent an infection in a mammal in need of such therapy.

In a preferred embodiment, the enhancer may be used to treat or prevent infections caused by gram positive or gram negative infections.

In a further preferred embodiment, the enhancer may be used to treat or prevent infections caused by Streptococci.

Other advantages of the present invention will become apparent from the ensuing detailed description taken in conjunction with the following illustrative drawings.

### BRIEF DESCRIPTION OF THE DRAWINGS

FIG. 1 shows the killing of 5 strains of S. pneumoniae with 1 U/ml Pal, Cpl-1 or a combination of both in 30 seconds (DCC 1335, open bar; DCC 1355, hatched; DCC 1420, stippled; DCC 1494, filled, DCC 1490, cross-hatched). The combination of the two enzymes shows more than additive killing on a logarithmic scale.

FIG. 2 shows time-kill curves for S. pneumoniae strains DCC 1355 (A) and DCC 1494 (B) with Pal (filled circles), Cpl-1 (open circles) and a combination of both (stars) at 0.25 [minimal inhibitory concentration (EC) each (12.5 U/ml Pal and Cpl-1 for DCC 1355, 12.5 U/ml Pal and 6.25 U/ml Cpl-1 for DCC 1494).

FIG. 3 shows an isobologram of the checkerboard synergy testing method, showing results for S. pneumoniae strains DCC 1490 and DCC 1355. For each well along the inhibitory line, enzyme concentrations (fractions of their MIC) were entered in an X/Y plot. Error bars show standard error of means. The two dashed lines illustrate theoretical curves.

### DETAILED DESCRIPTION OF THE INVENTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” include one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference in their entirety.

### DEFINITIONS

“Treatment” refers to the administration of medicine or the performance of medical procedures with respect to a patient, for either prophylaxis (prevention) or to cure the infirmity or malady in the instance where the patient is afflicted.

A “therapeutically effective amount” is an amount sufficient to decrease or prevent the symptoms associated with the bacterial infection.

A “prophylactically effective amount” is an amount sufficient to prevent a bacterial infection or the symptoms associated with the infection.

A “bacteriostatic” amount as used herein, is used to describe an amount of the combination of each of the synergistic enzymes, or a composition comprising the at least two, or the at least three, or the at least four, synergistic enzymes, sufficient to inhibit the growth of the infectious organisms.

A “bacteriocidal” amount as used herein, is used to describe an amount of the combination of each of the synergistic enzymes, or a composition comprising the at least two, or the at least three, or the at least four, synergistic enzymes, sufficient to kill the infectious organisms.

“Synergy” or “synergistic” amount or activity refers to the effects of a combination of antimicrobial agents (such as the lytic enzymes described herein), wherein the antimicrobial activity of the combination is greater than the sum of the activity of the individual antimicrobial agents.

“Peptidoglycan” is a thick rigid layer that is found in both gram positive and gram negative bacterial cells. It is composed of an overlapping lattice of two sugars that are crosslinked by amino acid bridges. The exact molecular makeup of these layers is species specific. The two sugars are N-acetyl glucosamine (NAG) and N-acetyl muramic acid (NAM). Attached to NAM is a side chain generally of four amino acids. The crossbridge is most commonly composed of L-alanine, D-alanine, D-glutamic acid, and diamino pimelic acid (DPA). The D-amino acids are different than the L-amino acids found in proteins. D-amino acids have the identical structure and composition as L-amino acids except that they are mirror images of the L amino acids. Most biological systems have evolved to commonly handle only the L form of compounds. Bacteria however use the D-aminoacids in their cell walls and have enzymes called racemases to convert between D and L forms. The NAM, NAG and amino acid side chain form a single peptidoglycan unit that can link with other units via covalent bonds to form a repeating polymer. The polymer is further strengthened by cross links between the D-glutamic acid of one unit and DPA of the next glycan tetrapeptide. In some gram positive microbes there is often a peptide composed of glycine, serine and threonine in between the crossbridges. The degree of cross-linking determines the degree of rigidity. In gram positive cells the peptidoglycan is a heavily cross-linked woven structure that wraps around the cell. It is very thick with peptidoglycan accounting for 50% of the weight of the cell and 90% of the weight of the cell wall. Electron micrographs show the peptidoglycan to be 20-80 nm thick. In gram negative bacteria the peptidoglycan is much thinner with only 15-20% of the cell wall being made up of peptidoglycan which is only intermittently cross-linked. In both cases the peptidoglycan can be thought of as a strong, woven mesh that holds the cell shape. It is not a barrier to solutes, the openings in the mesh are large and all types of molecules can pass through them. The cell wall is the site of action of many important antibiotics and antibacterial agents.

An “amidase” is an enzyme that catalyzes the hydrolysis of an amide to an acid and ammonium.

A “muramidase” is a glycosidase enzyme that hydrolyzes the bond between N-acetyl muramic acid and N-acetylglucosamine.

A “glucosaminidase” or “β-N-acetylglucosaminidase” is an enzyme that cleaves all non-reducing terminal β-linked N-acetylglucosamine.

“Lysozyme” is a muramidase that hydrolyzes the bond between N-acetyl muramic acid and N-acetyl glucosamine, thus cleaving an important polymer of the cell wall of many bacteria.

An “endopeptidase” refers to any of a large group of enzymes that catalyze the hydrolysis of peptide bonds in the interior of a polypeptide chain or protein molecule.

“Colonization” refers to the presence of bacteria within a persons body, however, the bacteria do not cause a disease.

General Description

In its broadest aspect, the present invention provides for a method of treating or preventing bacterial infections, comprising administering to a subject in need of such therapy a therapeutically effective or a prophylactically effective amount of a synergistic combination of bacteriophage derived lytic enzymes and a suitable carrier for delivery of the lytic enzymes to the site of infection. While an additive anti-microbial effect of the enzymes may have been predicted, it was not until the time of the present invention that the unexpected synergistic antimicrobial effect using at least two different lytic enzymes obtained from a bacteriophage was demonstrated.

The lytic enzymes useful for the prophylaxis or treatment of infections may be selected from the group consisting of an amidase, a muramidase, a glucosaminidase and an endopeptidase. A combination of any of the aforementioned enzymes may be useful for the methods described herein. While the preferred embodiment provides for the use of at least two of the lytic enzymes, another preferred embodiment provides for a combination of at least three of the enzymes described above. A yet further preferred embodiment provides for a combination of at least four of the enzymes described herein. Another preferred embodiment provides for pharmaceutical compositions for treating patients suffering from bacterial infections, or for preventing bacterial infections in patients susceptible to infections with bacteria having peptidoglycan in their cell wall. Anti-microbial compositions for sanitizing or decontaminating inanimate objects and surfaces comprising a combination of any of the lytic enzymes described herein are also objects of the present invention.

In a particular embodiment, Pal and Cpl-1, two purified bacteriophage lytic enzymes, were tested for their in vitro activity, alone and in combination, against several serotypes of Streptococcus pneumoniae, including penicillin-resistant strains. The enzymes demonstrated synergy in their ability to cleave the bacterial peptidoglycan and thus may be more efficient for the prevention and elimination of pneumococcal colonization.

The effect of the two bacteriophage lytic enzymes described herein provides a new mechanism for killing bacterial cells due to their combined synergistic lytic effect on the cell walls of both penicillin sensitive and penicillin resistant streptococci. This combination may play an important role in cleaving the peptidoglycan in bacterial cell walls and thus may be more efficient for the prevention and elimination of pneumococcal colonization. The findings in the present application suggest that a search for other combinations of this type of enzyme, or for mimics of the dual enzymatic and synergistic activity may prove to be a useful strategy for identifying a new class of antimicrobial agents and treatment modalities.

In a specific embodiment, the Cpl-1 expressing E. coli DH5α(pJML6) was constructed as follows: The Cpl-1 gene was amplified from Cpl-1 total phage DNA with a primer pair designed according to the published GenBank sequence number Z47794, flanked by XbaI and HindIII restriction sites, transcriptional start and stop codons, and a ribosomal binding site. Activity of the enzyme was also monitored by exposing S. pneumoniae strain DCC 1490 (serogroup 14), grown to log phase and resuspended in sterile saline, to an equal volume of serial 2-fold dilutions of the enzymes for 15 minutes in a microtiter plate. The reciprocal of the highest dilution that decreased the optical density by half (from a starting point of 0.5) was defined as the activity in U/ml. The specific activity of a freshly produced and purified batch for both enzymes is approximately 1 U/ug.

Another aspect of the invention provides for identification of small molecule mimics of the combination of Pal and Cpl-1 for use as antibacterial agents that kill both penicillin sensitive and penicillin resistant strains of streptococci, such as S. pneumoniae. In one specific embodiment, a method for identifying and testing the effect of a peptidomimetic of dual enzyme activity is provided herein and includes use of an assay such as the luciferin/luciferase assay system wherein one measures release of ATP from killed bacteria as light emitted in the presence of luciferin/luciferase.

Therapeutic Uses of the Invention

The antimicrobial activity of the synergistic lytic enzymes of the present invention advantageously permits treatment of bacterial infection in any animal, particularly mammals, and more particularly humans. Other animals that can be treated include, but are not limited to, pets (dogs, cats, rodents, ferrets, etc.); laboratory animals (rats, mice, rabbits, hamsters, guinea pigs, etc.); farm animals; and wild animals, e.g., in a zoo.

The synergistic lytic enzymes of the present invention may be used therapeutically to inhibit the growth of bacteria comprising cell walls that are susceptible to the activity of the synergistic lytic enzymes. The methods of this invention may also be applied prophylactically to prevent a patient from becoming sick after an exposure to a potentially infectious bacterium. Without being bound to a theory, this growth inhibition may occur by cleavage of the chemical bonds within the peptidoglycan structure of the bacterial cell wall. This cleavage may weaken the cell wall structure to physical and osmotic stresses, thereby increasing chances for cell wall rupture. This invention may include embodiments wherein bacterial infections of the respiratory tract, nasal cavity, the throat, the mouth and the sinus cavities are treated. Other methods which accomplish delivery of the synergistic lytic enzyme-containing compositions to any site of infection are also suitable and are possible embodiments of the present invention.

The at least two lytic enzymes of the present invention can also be used successfully prophylactically on medical instruments, e.g., as a sterilization agent. When applied to catheters, stents, artificial joins, pins, and other implanted devices, the synergistic lytic enzymes prevent development of infections. In certain applications, the lytic enzymes may be implanted by including recombinant cells that produce the protein in the device, e.g., for coronary or peripheral arterial shunts.

In a preferred embodiment, the foregoing synergistic lytic enzymes provide for prophylaxis and methods of treatment of pneumonia, particularly in a hospital setting.

Another aspect of the invention provides for the use of Pal and Cpl-l enhancers, mimics or modulators in prevention of bacterial cell growth in vitro and in vivo. One embodiment of the invention features broad spectrum use of the enhancers, mimics or modulators to prevent growth of gram positive and gram negative bacteria, as well as inhibition of growth of other strains of bacteria. The enhancers of Pal and Cpl-1 activity are envisioned to be small molecules, peptides, polypeptides or mimics thereof

The invention provides for treatment or prevention of various diseases and disorders by administration of a therapeutic agent. Such agents include but are not limited to: agents that modulate the activity of Pal and Cpl-1 activity, and related analogs, derivatives, and fragments thereof. Such agonists may include small molecule or peptide mimics of the Pal and Cpl-1 combination.

In one embodiment wherein enhancement of Pal and Cpl-1 is desirable, one or more enhancers or mimics, each specifically binding to the Pal and/or the Cpl-1, are administered alone or in combination with one or more additional therapeutic compounds or treatments. In a preferred embodiment, a Pal and Cpl-1 enhancer or mimic is administered to a human subject for therapy (e.g. to ameliorate symptoms or to retard onset or progression) of bacterial infections.

Therapeutic and Prophylactic Compositions and Their Use

The invention provides methods of treatment comprising administering to a subject an effective amount of an agent of the invention. In a preferred aspect, the compound is substantially purified (e.g., substantially free from substances that limit its effect or produce undesired side-effects). The patient or subject is preferably an animal, including but not limited to animals such as monkeys, cows, pigs, horses, chickens, cats, dogs, etc., and is preferably a mammal, and most preferably human. In one specific embodiment, a non-human mammal is the subject. In another specific embodiment, a human mammal is the subject.

Various delivery systems are known and can be used to administer a compound of the invention, e.g., encapsulation in liposomes, microparticles, or microcapsules. Methods of introduction can be enteral or parenteral and include but are not limited to intradermal, intramuscular, intraperitoneal, intravenous, subcutaneous, intranasal, epidural, topical and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. In a specific embodiment, it may be desirable to administer the pharmaceutical compositions of the invention locally to the area in need of treatment, such as topical use on the skin; any suitable method known to the art may be used.

Another aspect of the invention provides for pharmaceutical compositions comprising purified Pal and Cpl-1 or enhancers of the activity of these two enzymes for therapeutic use in treatment and/or prevention of bacterial infections. One embodiment features treatment of a wide range of infections including those caused by gram positive, gram negative or mycobacterial infection with pharmaceutical compositions containing acceptable carriers and excipients. Moreover, a further embodiment may include a pharmaceutical composition designed for use in topical treatment of bacterial infections. Another embodiment may include a pharmaceutical composition designed for use in treatment of systemic infections, or infections that are non-responsive to other antibiotic modalities.

Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene; glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

The present invention thus provides a method of enhancing the antimicrobial effects of bacteriophage derived lytic enzymes, which, when used alone, show the benefit of killing certain bacteria through their deleterious effect on the peptidoglycan in the cell wall. When administering an amidase, such as Pal, together with a lysozyme, such as Cpl-1, the effects of this combination are synergistic rather than additive, as shown in the present application. Thus, the compositions of the instant application provide for a combined preparation for simultaneous or sequential administration for the treatment of or the prevention of bacterial infections.

Preferably, this administration of the synergistic lytic enzymes is repeated over a period of several or multiple days.

In a preferred embodiment, the amidase (eg. Pal) is used concurrently with either a muramidase, such as lysozyme (eg. Cpl-1), or a glusosaminidase, or an endopeptidase, although sequential therapy may be envisioned as well.

The amount of amidase (eg. Pal) to be used as described in the present invention varies according to the degree of the effective amount required for treating specific bacterial infections. The amount of lysozyme (eg. Cpl-1) or glucosaminidase or endopeptidase to be used as described in the present invention also varies according to the degree of the effective amount required for treating specific bacterial infections. When combined together in the same composition, the preferred amount of each will be titrated to achieve the desired effective dose level. Procedures for titrating the most effective dose level of each enzyme are known to those skilled in the art.

The bacterial infections for which a composition containing Pal and Cpl-1 is a particularly suitable treatment include infections caused by streptococcus, including S. pneumoniae, although the invention should not be construed to be limited to one particular organism.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Liquid form preparations include solutions, suspensions and emulsions. As an example may be mentioned water or water-propylene glycol solutions for parenteral injection.

Also included are solid form preparations which are intended to be converted, shortly before use, to liquid form preparations for either oral or parenteral administration. Such liquid forms include solutions, suspensions and emulsions.

The lytic enzymes of the present invention may also be deliverable transdermally. The transdermal compositions can take the form of creams, lotions, aerosols and/or emulsions and can be included in a transdermal patch of the matrix or reservoir type as are conventional in the art for this purpose.

Preferably, the pharmaceutical preparation is in unit dosage form. In such form, the preparation is subdivided into unit doses containing appropriate quantities of the active component, e.g., an effective amount to achieve the desired purpose.

The actual dosage employed may be varied depending upon the requirements of the patient and the severity of the condition being treated. Determination of the proper dosage for a particular situation is within the skill of the art. For convenience, the total daily dosage may be divided and administered in portions during the day, if desired.

The combination of lytic enzymes may be administered using conventional techniques such as oral administration. Intravenous administration is preferable for a continuous dosing therapy regimen. Oral administration can be utilized for a repeat dosing regimen.

The amidase can be administered separately prior to, or concurrent with, the lysozyme. Where it is desirable to do so, both the amidase and lysozyme can be combined into a unit dosage form to facilitate patient dosing. Such combination dosage forms may be in any of the above-described dosage forms, but, as noted above, are preferably in oral or intravenous forms.

The amidase and the lysozyme can be packaged in a kit form. In such a kit, the amidase and the lysozyme would be individually formulated into particular dosage forms for the particular route of administration, and contain instructions for the administration of the contents. In a typical embodiment for oral formulation, such a kit may be in the form of a blister package with separately formulated oral dosage forms of the amidase and the lysozyme.

Any necessary adjustments in dose can be readily made to meet the treatment requirements of the individual patient and adjusted accordingly by the skilled practitioner.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the compound of the invention which will be effective in the treatment of infectious diseases wherein the bacteria contain the Pal and Cpl-1 enzymes can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

In a specific embodiment, it may be desirable to administer the pharmaceutical compositions of the invention locally to the area in need of treatment; this may be achieved, for example, and not by way of limitation, by local infusion during surgery, by topical application, by injection, by means of a catheter, or by means of an implant, said implant being of a porous, non-porous, or gelatinous material, including membranes, such as sialastic membranes, or fibers or co-polymers such as Elvax (see Ruan et al, 1992, Proc Natl Acad Sci USA, 89:10872-10876). In one embodiment, administration can be by direct injection by aerosol inhaler.

In another embodiment, the Pal and Cpl-1 combination or enhancer compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the Pal and Cpl-1 combination or enhancer compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, J. (1983) Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, i.e., the airways, thus requiring only a fraction of the systemic dose (see, e.g. Goodson, in Medical Applications of Controlled Release (1984) supra, vol. 2, pp. 115-138). Other suitable controlled release systems are discussed in the review by Langer (1990) Science 249:1527-1533.

Effective Dose

The lytic enzymes described herein can be administered to a patient at therapeutically effective doses to treat certain diseases or disorders. A therapeutically effective dose refers to that amount of a therapeutic sufficient to result in a healthful benefit in the treated subject. A prophylactically effective dose refers to that amount of a prophylactic sufficient to result in prevention of a disease in a patient.

The precise dose of the therapeutic embodied by this invention, to be employed in the formulation, will depend on the route of administration, and the nature of the patient's disease, and should be decided according to the judgment of the practitioner and each patient's circumstances according to standard clinical techniques. The term “inhibit” or “inhibition” means to reduce by a measurable amount. Experimental evidence of inhibition may include observing the elimination of a bacterial infection in an animal model. Effective doses may thus be extrapolated from dose-response curves derived from animal model test systems.

Toxicity and therapeutic efficacy of the lytic enzymes can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutics that exhibit large therapeutic indices are preferred. While therapeutics that exhibit toxic side effects can be used, care should be taken to design a delivery system that targets such compounds to the site of affected tissue in order to minimize potential damage to uninfected cells and, thereby, reduce side effects.

The data obtained from the in vitro bacterial growth inhibition and cell lysis assays and animal studies can be used in formulating a range of dosage for use in humans. The dosage of lytic enzymes containing compositions lies preferably within a range of concentrations that include the ED₅₀ with little or no toxicity. The dosage can vary within this range depending upon the dosage form employed and the route of administration utilized. For any composition used in the method of the invention, the therapeutically effective dose can be estimated initially from in vitro bacterial growth inhibition and cell lysis assays. A dose can be formulated in animal models to achieve a lytic enzyme concentration range that includes the IC₅₀ (i.e., the concentration of the test compound which achieves a half-maximal inhibition of symptoms). Such information can be used to more accurately determine useful doses in humans. The efficacy of a particular dosage in eliminating, for example, a bacterial throat infection may be measured by a traditional throat culture swab assay. Any method which provides data as to the number of bacteria present at the site of infection would be suitable for these purposes.

In order to be effective, the enzymes should be present in an amount sufficient to provide an effective number of enzyme units in contact with the oral cavity or respiratory tract. Having too few enzyme units in contact with the oral cavity or the respiratory tract, even over a long period of time, will not produce as beneficial an effect as desired. Hence, any dosage form employed should provide for an approximate minimum number of units for the amount of time that the dosage will provide enzyme to the oral cavity or the respiratory tract. The concentration of the active units of enzymes believed to provide for an effective amount or dosage of enzymes may be in the range of from about 100 units to about 100,000 units in the environment of the nasal and oral passages. Within that broader range, dosages of from about 100 units to about 10,000 units are believed to be acceptable. Such units can be contained in smaller volumes of carrier such as liquid or saliva, e.g. 1 ml or less (e.g. in the case of a lozenge) or can be contained in larger dosage volumes such as a gargle of several mls. Generally, therefore, larger volumes of carrier will require a greater total number of units to achieve an effective concentration of active enzyme. Hence, acceptable concentrations can be from about 100 units/ml to about 100,000 units/ml of fluid in the environment of the nasal or oral passages. Within this range, concentrations from about 100 units/ml to about 10,000 units/ml are acceptable.

In practice, therefore, the time exposure to the active enzyme units likely will influence the desired concentration of active enzyme units employed in the dosage per ml. For example, carriers that are considered to provide prolonged release (certain nasal sprays, lozenges and encapsulated enzyme) could provide a lower concentration of active enzyme units per ml, but over a longer period of time. Conversely, a shorter duration treatment (e.g., a gargle) could provide a higher concentration of active enzyme units per ml. Any dosage form containing sufficient lytic enzyme to provide effective concentrations of active enzyme at the site of infection or to provide a sufficient prophylactic effect are well within the bounds of routine experimentation and therefore, well within the scope of the instant invention.

Compositions for Sanitizing or Decontaminating Inanimate Porous or Non-porous Objects

In yet another embodiment, a composition is envisioned for sanitizing or decontaminating inanimate non-porous or porous objects using a bactericidal or bacteriostatically effective amount of the combination of the at least two synergistic bacteriophage lytic enzymes. The composition of the present invention for this particular use may be employed for reducing the microbial population of surgical instruments, surgical gowns, examination areas, and other medical or veterinarian tools or instruments.

It is also envisioned that the composition of the present invention may be employed for disinfecting dental instruments. Thus, the composition containing Pal and Cpl-1 can prevent a potential infection which may arise from the use of instruments or equipment that are contaminated with bacteria which may be susceptible to the killing activity of Pal and Cpl-1. Furthermore, the amount of each lytic enzyme for use in the composition would be determined through standard methods of titration for anti-microbial activity known to those skilled in the art.

Although the antibacterial property of lysozymes in general has been well documented (Procter et al in CRC Crit. Reviews in Food Science and Nutrition, 1988, 26(4):359-395), the synergistic anti-microbial effect of lysozyme, in particular Cpl-1, in combination with bacteriophage amidases, such as Pal, has not been disclosed. It is with regard to this unexpected finding that the present invention is directed.

The compositions of the present invention for sanitizing or for disinfectant use may include other additives. Examples of such additives include one or more anti-microbial agents, including other lytic enzymes, or bacteriocins, or chelating agents. Other agents that enhance the permeability of the antibacterial agents or lytic enzymes are also contemplated.

Furthermore, the composition may include other agents that function to stabilize the bacteriophage derived lytic enzymes. It is envisioned that the composition may be provided in liquid form, as a spray, as a gel, as a foam, or as a powder.

The compositions of the present invention may include surfactants such as an alkyl glycoside or an alkenyl glycoside, an alkoxylated alcohol, an alkoxylated carboxylic acid, a sorbitan ester, a polyethoxylated derivative of a sorbitan ester, a polyglycerol ester, a sucrate, an ester of fatty acid with a polyalcohol and/or a polyalkylene glycol. Other additives such as thickeners, preservatives, foaming agents may also be present in the composition.

In order to accelerate treatment of the infection, the administration of a second therapeutic agent may be an embodiment of this invention. The second therapeutic agent may be administered to the patient as a part of the lytic enzyme composition or in the form of a separate composition which comprises only said second therapeutic agent. A non-limiting exemplary list of suitable second therapeutic agents includes penicillin, synthetic penicillins bacitracin, methicillin, cephalosporin, polymyxin, cefaclor. Cefadroxil, cefamandole nafate, cefazolin, cefixime, cefinetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone moxalactam, cefuroxime, cephalexin, cephalosporin C, cephalosporin C sodium salt, cephalothin, cephalothin sodium salt, cephapirin, cephradine, cefuroximeaxetil, dihydratecephalothin, moxalactam, loracarbef mafate, chelating agents and any combinations thereof in amounts which are effective to further enhance the therapeutic effect of the lytic enzyme. Furthermore, antifingal compositions may be coadministered with the lytic enzyme containing compositions of this invention (separately or within the same composition as the lytic enzyme) Such antifungal agents may include Amphotericin B, Carbol-Fuchsin, Ciclopirox, Clotrimzole, Econazole, Haloprogin, Ketoconazole, Mafenide, Miconazole, Naftifine, Nystatin, Oxiconazole Silver, Sulfadiazine, Sulconazole, Terbinatine, Tioconazole, Tolnaftate, Undecylenic acid, flucytosine, miconazole, or others.

Compositions of the present invention may further comprise antiviral agents. Such antiviral agents may include zinc containing substances, such as zinc gluconate. Furthermore anesthetic agents may be included in the compositions of the present invention. Suitable anaesthetics may include aspirin, acetaminophen, phenol, benzocaine, diphenhydramine, kaolin-pectin and Xylocaine. Furthermore, decongestants and antihistamines may be included in compositions of the invention. Suitable decongestants may include pseudoephedrine, phenylpropanolamine and phenylephrine; antihistamines may include brompheniramine or chlorpheniramine.

Screening Assays

The invention provides methods for identifying agents (e.g., chemical compounds, carbohydrates, proteins, peptides, antibodies or nucleotides) that have the ability to cleave the peptidoglycan found in the cell walls of various bacteria. The invention also provides methods of identifying agents, candidate compounds or test compounds that specifically mimic the synergistic activity of the two bacteriophage derived lytic enzymes described herein. Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to-peptide, non-peptide oligomer or small molecule libraries of compounds (Lam, 1997, Anticancer Drug Des. 12:145; U.S. Pat. No. 5,738,996; and U.S. Pat. No. 5,807,683, each of which is incorporated herein in its entirety by reference).

In one embodiment, agents that mimic the action of Pal and Cpl-1 are identified in a cell-based assay system. In accordance with this embodiment, cells expressing a combination of both lytic enzymes, or active fragments thereof, are contacted with a candidate compound or a control compound and the ability of the candidate compound to compete with the combination of Pal and Cpl-1 for binding to bacterial peptidoglycan is determined. If desired, this assay may be used to screen a plurality (e.g., a library) of candidate compounds. The cell, for example, can be of prokaryotic origin (e.g., S. pyogeties or S. aureus), and may contain Pal and Cpl-1 peptides or polypeptides, fragments, or related polypeptide thereof In some embodiments, the Pal and Cpl-1 enzymes or polypeptides, fragments, or related polypeptides thereof or the candidate compound is labeled, for example with a radioactive label (such as ³²P, ³⁵S or ¹²⁵I or a fluorescent label (such as fluorescein isothiocyanate, rhodamine, phycoerythrin, phycocyanin, allophycocyanin, o-phthaldehyde or fluorescamine) to enable detection of an interaction between Pal and Cpl-1 and a candidate compound. The ability of the candidate compound to interact directly or indirectly with the Pal and Cpl-1 can be determined by methods known to those of skill in the art. For example, the interaction can be determined by flow cytometry, a scintillation assay, immunoprecipitation or Western blot analysis.

In another embodiment, agents interact with (i.e., bind to) Pal and Cpl-1 in a cell-free assay system. In accordance with this embodiment, Pal and Cpl-1 are contacted with a candidate compound or a control compound and the ability of the candidate compound to interact with the Pal and Cpl-1 is determined. If desired, this assay may be used to screen a plurality (e.g. a library) of candidate compounds. In one embodiment, the Pal and Cpl-1 are first immobilized, by, for example, contacting the enzymes with an immobilized antibody which specifically recognizes and binds either or both Pal and Cpl-1, or by contacting a purified preparation of the Pal and Cpl-1 with a surface designed to bind proteins. The Pal and Cpl-1 may be partially or completely purified (e.g., partially or completely free of other polypeptides) or part of a cell lysate. (0106 In another embodiment, a cell-based assay system is used to identify agents that bind to or modulate the activity of the Pal and Cpl-1, or a biologically active portion thereof. In a primary screen, a plurality (e.g., a library) of compounds are contacted with cells that naturally express Pal and Cpl-1 in order to identify compounds that modulate the activity of both enzymes. The ability of the candidate compound to modulate the activity of both enzymes can be determined by methods known to those of skill in the art, including without limitation, flow cytometry, a scintillation assay, immunoprecipitation and western blot analysis.

In another embodiment, agents that competitively interact with (i.e., bind to) Pal and Cpl-1 are identified in a competitive binding assay. In accordance with this embodiment, cells containing Pal or Cpl-1 are contacted with a candidate compound and a compound known to interact with the Pal and Cpl-1 or prevent the activity of Pal and Cpl-1; the ability of the candidate compound to competitively interact with Pal and Cpl-1 or to competitively prevent activity of Pal and Cpl-1 is then determined. Alternatively, agents that competitively interact with (i.e., bind to) Pal and Cpl-1 or competitively prevent the activity of Pal and Cpl-1 are identified in a cell-free assay system by contacting the Pal and Cpl-1 or components able to form an active Pal and Cpl-1 combination with a candidate compound and a compound known to interact with or prevent the activity of Pal and Cpl-1. As stated above, the ability of the candidate compound to interact with Pal and Cpl-1 or prevent the activity of Pal and Cpl-1 can be determined by methods known to those of skill in the art. These assays, whether cell-based or cell-free, can be used to screen a plurality (e.g., a library) of candidate compounds.

In another embodiment, agents that modulate (i.e., up-regulate or down-regulate) the activity of Pal and Cpl-1 are identified by contacting cells (e.g., cells of prokaryotic origin) containing the components capable of forming an enzymatically active synergistic combination with a candidate compound or a control compound (e.g., phosphate buffered saline (CBS)) and determining the activity of the Pal and Cpl-1 combination. The level of Pal and Cpl-1 activity in the presence of the candidate compound is compared to the level of activity in the absence of the candidate compound (e.g., in the presence of a control compound). The candidate compound can then be identified as a modulator of the formation of the Pal and Cpl-1 synergistic combination based on this comparison. For example, when presence of an enzymatically active combination of Pal and Cpl-1 is significantly greater in the presence of the candidate compound than in its absence, the candidate compound is identified as a stimulator of Pal and Cpl-1 activity. Alternatively, when presence of an enzymatically active Pal and Cpl-1 is significantly less in the presence of the candidate compound than in its absence, the candidate compound is identified as an inhibitor of Pal and Cpl-1 activity. Such screening method is also envisioned for identifying agents capable of enhancing the activity of Pal and Cpl-1, comprising:

(a) preparing purified Pal and Cpl-1 ;

(b) contacting the Pal and Cpl-1 to a bacteria having radioactively labeled peptidoglycan in the cell wall in the presence or absence of a test compound under conditions which allow binding to the peptidoglycan; and

(c) determining the amount of peptidoglycan cleavage, wherein an agent capable of enhancing Pal and Cpl-1 activity is identified when the release of radioactivity is enhanced in the presence but not the absence of the agent.

In another embodiment, agents that modulate the activity of an enzymatically active Pal and Cpl-1 combination are identified by contacting a preparation containing the Pal and Cpl-1 enzymes, or cells (e.g., prokaryotic) forming an enzymatically active Pal and Cpl-1 combination with a test compound or a control compound and determining the ability of the test compound to modulate (e.g., stimulate or inhibit) the activity of the Pal and Cpl-1 combination. The activity of the Pal and Cpl-1 combination can be assessed in a number of ways, known to those skilled in the art.

In another embodiment, agents that modulate (i.e., up-regulate or down-regulate) the activity of Pal and Cpl-1 combination are identified in an animal model. Examples of suitable animals include, but are not limited to, mice, rats, rabbits, monkeys, guinea pigs, dogs and cats. Preferably, the animal used represents a model of disease or infection, such as that caused by bacteria. Examples of such infections may be those caused by Streptococcus, Staphylococcus, or Mycobacteria.

In accordance with this embodiment, the test compound or a control compound is administered (e.g., topically, orally, rectally or parenterally such as intraperitoneally or intravenously) to a suitable animal and the effect on the activity of Pal and Cpl-1 is determined. Changes in the activity of Pal and Cpl-1 can be assessed by any suitable method described above, based on the present description.

This invention further provides novel agents identified by the above-described screening assays and uses thereof for treatments as described herein.

Assays for Therapeutic Compounds

The present invention also provides for assays for use in discovery of pharmaceutical products in order to identify or verify the efficacy of compounds for treatment or prevention of bacterial infections. In one embodiment, agents can be assayed for their ability to inhibit bacterial growth in vitro or in vivo. Compounds able to enhance Pal and Cpl-1 activity in vitro can be further tested for anti-bacterial activity in experimental animal models of infectious disease and can be used as lead compounds for further drug discovery, or used therapeutically.

In various embodiments, in vitro assays can be carried out with cells that harbor the Pal and Cpl-1 enzymes and that are representative of the bacterial cell type involved in a subject's disease, to determine if a compound has a desired effect upon such bacterial cell types. In one embodiment, the cells are Streptococcus. In a preferred embodiment, the cells are Streptococcus pneumoniae.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used. In one embodiment, test compounds that modulate the activity of enzymatically active Pal and Cpl-1 combinations are identified in non-human animals (erg., mice, rats, monkeys, rabbits, and guinea pigs), preferably non-human animal models for infectious diseases. In accordance with this embodiment, a test compound or a control compound is administered to the animals, and the effect of the test compound on Pal and Cpl-1 levels or activity is determined in the bacterial organism obtained from the infected animal. A test compound that alters the level or activity of Pal and Cpl-1 combinations can be identified by comparing the level of the selected Pal and Cpl-1 enzymes in a bacterial culture obtained from an animal or group of animals treated with a test compound with the level of the Pal and Cpl-1 in a bacterial culture obtained from an animal or group of animals treated with a control compound.

In yet another embodiment, test compounds that modulate the level or activity of Pal and Cpl-1 are identified in human subjects having an infection associated with bacteria that contain the Pal and Cpl-1 enzymes. -In accordance with this embodiment, a test compound or a control compound is administered to the human subject, and the effect of a test compound on either reduction in spread of the microbial infection, elimination of the bacterial infection or amelioration of symptoms associated with the infection is determined by methods known in the art.

### EXAMPLES

The following examples are put forth so as to provide those of ordinary skill in the art with a complete disclosure and description of how to isolate and use the novel class of enzymes described herein, and to provide a suitable means for identifying and assaying the synergistic effects of this class of enzymes and development of pharmaceutical compositions for therapeutic use, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Bacterial Strain and Culture

E. coli DH5α(pMSP11) expressing Pal and double-stranded DNA from the pneumococcal phage Cpl-1 were used in the present studies (Sheehan et al. 1997, Mol. Microbiol. 25(4): 717-725). The Cpl-1 expressing E. coli DH5t(pJML6) was constructed as follows: The Cpl-1 gene was amplified from Cpl-1 total phage DNA with a primer pair designed according to the published GenBank sequence number Z47794, flanked by XbaI and HindIII restriction sites, transcriptional start and stop codons, and a ribosomal binding site. To use the same powerful expression system as for Pal, which is based on the construct pIN-IIIA and contains a double promoter, we digested the plasmid pMSP11 with XbaI and HindIII, which removes the entire inserted Pal gene (Masui et al, 1984, Bio/Technology:81-85, Sheehan et al. 1997, Mol. Microbiol. 25(4): 717-725). The PCR product was subcloned to pMSP11 using the XbaI and HindIII recognition sites to produce the pJML6 construct. E. coli DH5a(pMSP11) and DH5α(pJML6) were grown in LB broth and induced with lactose overnight. Harvest and purification of the enzymes are described elsewhere (Loeffler et al, 2001, Science 294(5549):2170-2172).

Enzyme Assay

Both enzymes were stored and assayed in 50 mM phosphate buffer (pH 7.0) containing 1 mM DTT and 1 mM EDTA (hereafter called enzyme buffer). Activity was measured by exposing S. pneumoniae strain DCC 1490 (serogroup 14), grown to log-phase and resuspended in sterile saline, to and equal volume of serial 2-fold dilutions of the enzymes for 15 minutes in a microtiter plate. The reciprocal of the highest dilution that decreased the optical density by half (from a starting point of 0.5) was defined as the activity in U/ml. The specific activity of a freshly produced and purified batch for both enzymes is approximately 1U/jig.

### Example 2

### Time Kill Experiments-Short Exposure

Time-kill experiments were conducted with very short exposures, since the killing with both enzymes can be observed within seconds. Moreover, it is envisioned that a typical application of compositions containing a combination of both enzymes in the nasopharynx would be unique and short. Mid-log-phase cultures of S. pneumoniae strains DCC 1355, DCC 1490, DCC 1494, DCC 1335, and DCC 1420, (serotypes 19, 14, 14, 9V, 23F and, the latter 3 highly penicillin-resistant) were pelleted and resuspended to an absorbance at 600 nm of 1.0 (approximately 10⁹ CFU/ml). 150 μl of Pal or Cpl-1 at a final concentration of 1 U/ml, or a mixture of both at a concentration of 0.5 U/ml each was added to 150 μl of the bacterial solution. Colony counts were performed after 30 seconds and 10 minutes and compared to a control exposed to enzyme buffer only, by serially diluting a 10 μl aliquot in saline and plating on 5% Columbia blood agar (CBA), with a detection limit of 10⁴ CFU/ml.

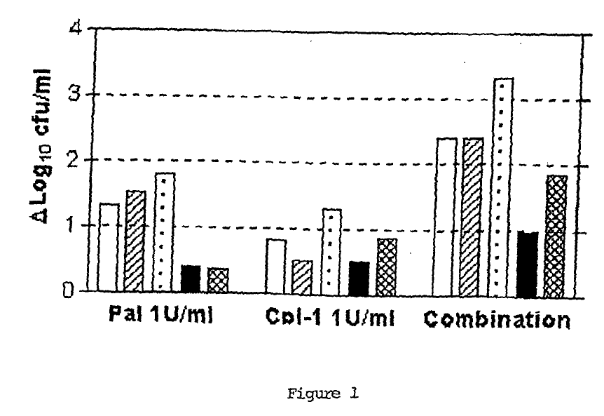

In 30 seconds 1 U/ml Pal reduced the bacterial titer of the 4 strains by Log₁₀ CFU/ml (median range) 1.34 (0.38-1.81), while Cpl-1 at 1 U/ml reduced the titers by log₁₀ CFU/ml 0.83 (0.52-1.31). The combination of both enzymes reduced the titers by log₁₀ CFU/ml 2.40 (0.98-3.34) (FIG. 1). After 10 minutes, reduction was log₁₀ CFU/ml (median range) 1.99 (0.73-2.54), 1.44 (1.32-2.65), and 3.15 (2.50-5.28), for Pal, Cpl-1 and the combination respectively. In other words, mixing 0.5 U/ml of each enzyme increased killing efficacy by log₁₀ 1.07 to 131 (median, 30s and 10 min), compared to Pal alone and by log₁₀ 1.58 to 1.72 (30s and 10 min), compared to Cpl-1 alone. Using one-way ANOVA and the Bonferroni post-test for the comparison of the three treatment groups, the killing efficacy of the combination of enzymes was always significantly higher than that of both single enzymes (p<0.05), at both time points, except for strain DCC 1494 at 30 seconds.

### Example 3

### Time Kill Experiments-Long Exposure

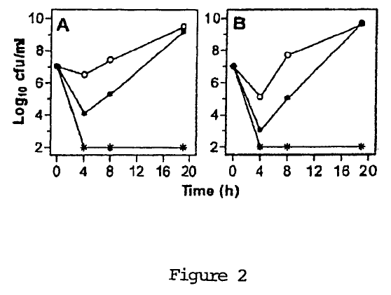

Time-kill studies with longer exposure (up to 19 h) were performed using only strains DCC 1355 and DCC 1494. Bacteria were grown in cation-adjusted Müller-Hinton broth (CAMHB) with 2.5% lysed horse blood to mid-log-phase and resuspended in fresh medium at a titer of 1×10⁷ CFU/ml. Pal, Cpl-1 or a combination of both were added at 0.25 MIC (12.5 U/ml of Pal and Cpl-1 for strain DCC 1355, 12.5 U/ml of Pal and 6.25 U/ml of Cpl-1 for strain DCC 1494). Samples were taken at 0, 4, 8 and 19 hours, serially diluted and plated on CBA for titer determination, with a detection limit of 100 CFU/ml. The usual definition of a titer reduction of the combination ≧2 log₁₀ greater than that of the single most active agent was used for determination of synergy. FIG. 2 illustrates the results, which show synergy for both strains.

### Example 4

### Determination of Minimal Inhibitory Concentration

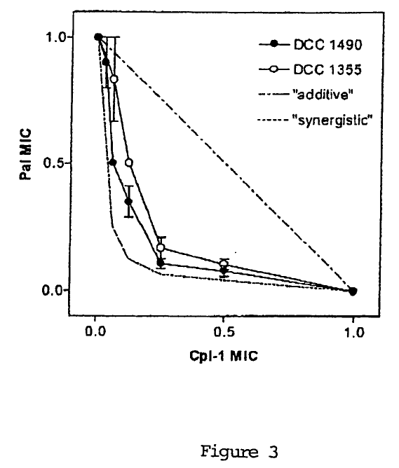

The well-described checkerboard broth microdilution method, which allows for the concurrent determination of the minimal inhibitory concentration (NIC) of each agent tested (Eliopoulos et al. 1991, Antimicrobial Combinations, V. Lorian Ed., Williams & Wilkins, Baltimore, Md.), was then applied. Testing was performed repeatedly with all 5 strains described above in CAMHB supplemented with 5% lysed horse blood, with a final inoculum of 5×10⁴ CFU per well. Both enzymes were assayed at concentrations between 200 and 3.125 U/ml. Plates were incubated at 37° C. for 18 h in ambient air and examined visually for growth using a reflective viewer. The fractional inhibitory concentration index (ΣFIC) [i.e., the sum of the fractions of both MICs that showed inhibition) was calculated. A ΣFIC of ≦0.5 was interpreted as synergy. The MICs were 50-200 U/ml for Pal and 25-50 U/ml for Cpl-1 in all tested strains. Transcription, of the enzyme concentrations along the inhibitory line on the microtiter plate into an isobologram revealed curves with a characteristic shape of synergy. The results of strains DCC 1490 and DCC 1355 are shown in FIG. 3. The ΣFIC for all strains was ≦0.5.

The combination of the two lytic bacteriophage enzymes Pal and Cpl-1 appears to have synergistic activity on several S. pneumoniae strains, including those that are penicillin-resistant, in vitro. This positive interaction could either to be due to the increased access of these enzymes to the respective cleavage sites or to the enhanced destructive effect of a two-dimensional digestion in the three-dimensional peptidoglycan.

1. A method of treating or preventing bacterial infections, comprising administering to a subject in need of such therapy a therapeutically effective amount of a synergistic combination of:
(a) at least two bacteriophage derived lytic enzymes; and
(b) a suitable carrier for delivery of the lytic enzymes to the site of infection.

2. The method of claim 1, wherein said at least two bacteriophage derived lytic enzymes are selected from the group consisting of an amidase, a muramidase, an endopeptidase, a glucosaminidase and combinations thereof.

3. The method of claim 1, wherein said at least two bacteriophage derived lytic enzymes are an amidase and a muramidase.

4. The method of claim 3, wherein the muramidase is lysozyme and the amidase is Pal.

5. The method of claim 4, wherein the lysozyme is Cpl-1.

6. The method of claim 1, wherein the lytic enzymes are selected from the group consisting of Pal and Cpl-1.

7. The method of claim 1, wherein the bacterial infections are caused by Streptococcus pneumoniae.

8. The method of claim 1, wherein the lytic enzymes decrease the occurrence or severity of local and systemic pneumococcal disease.

9. The method of claim 1, wherein the lytic enzymes prevent or eliminate pneumococcal colonization.

10. The method of claim 1, wherein said at least two lytic enzymes are isolated from the phage of disease causing bacteria, and wherein said disease causing bacteria are gram positive bacteria.

11. A method of treating or preventing bacterial infections, comprising administering to a subject in need of such therapy a therapeutically effective amount of bacteriophage derived lytic enzymes selected from the group consisting of an amidase, a muramidase, an endopeptidase, a glucosaminidase, and synergistic combinations thereof.

12. The method of claim 10, wherein the gram positive bacteria is Streptococcus.

13. The method of claim 12, wherein the Streptococcus is Streptococcus pneumoniae.

14. The method of claim 11, wherein the amidase is Pal and the muramidase is Cpl-1.

15. A pharmaceutical composition comprising:
(a) at least two therapeutically effective synergistic bacteriophage derived lytic enzymes; and
(b) a carrier suitable for delivery of the lytic enzymes to the site of infection.

16. The composition of claim 15, wherein said at least two bacteriophage derived lytic enzymes are selected from the group consisting of an amidase, a muramidase, an endopeptidase, a glucosaminidase and combinations thereof.

17. The pharmaceutical composition of claim 16, wherein the amidase is Pal and the muramidase is Cpl-1.

18. A screening method for identifying agents capable of enhancing the activity of Pal and Cpl-1, comprising:
(a) preparing purified Pal and Cpl-1;
(b) contacting the Pal and Cpl-1 to a bacteria having radioactively labeled peptidoglycan in the cell wall in the presence or absence of a test compound under conditions which allow binding to the peptidoglycan; and
(c) determining the amount of peptidoglycan cleavage, wherein an agent capable of enhancing Pal and Cpl-1 activity is identified when the release of radioactivity is enhanced in the presence but not the absence of the agent.

19. An anti-microbial composition for sanitizing or decontaminating porous or non-porous surfaces comprising at least two bacteriophage derived synergistic lytic enzymes.

20. The composition of claim 19, wherein said at least two bacteriophage derived synergistic lytic enzymes are selected from the group consisting of an amidase, a muramidase, an endopeptidase, a glucosaminidase and combinations thereof.

21. The composition of claim 19, wherein said at least two bacteriophage derived lytic enzymes are an amidase and a muramidase.

22. The composition of claim 21, wherein the muramidase is lysozyme and the amidase is Pal.

23. The composition of claim 22, wherein the lysozyme is Cpl-1.

24. A method for decontaminating inanimate surfaces suspected of containing infectious bacteria comprising treatment of said surfaces with a bacteriocidal or bacteriostatically effective amount of the composition of claim 19.

25. (canceled)

26. (canceled)

27. (canceled)

28. (canceled)

29. (canceled)
